(19) United States
(12) Patent Application Publication
(10) Pub. No.: US 2017/0311569 A1
(43) Pub. Date: Nov. 2, 2017

(54) GENTLE ANIMAL COLLAR FOR USE WITH ALERT AND PREVIOUSLY CANNULATED ANIMALS, AND ASSOCIATED SYSTEMS AND METHODS

(71) Applicant: University of Iowa Research Foundation, Iowa City, IA (US)

(72) Inventors: Brandon Rea, Iowa City, IA (US); Aaron Fairbanks, Iowa City, IA (US); Andrew Russo, Iowa City, IA (US); Levi Sowers, Iowa City, IA (US)

(21) Appl. No.: 15/583,521

(22) Filed: May 1, 2017

Related U.S. Application Data
(60) Provisional application No. 62/329,712, filed on Apr. 29, 2016.

(51) Int. Cl. A01K 15/04; A61B 5/00; A61M 5/158; A61F 5/37

(52) U.S. Cl. CPC A01K 15/04; A61F 5/37; A61B 5/0077; A61M 5/158; A61B 2503/40; A61B 2503/42; A61M 2250/00

(57) ABSTRACT

The disclosed apparatus, systems and methods relate to various devices, systems and methods for gently restraining an unanesthetized laboratory animal, particularly for facial recognition applications. In some applications, the laboratory animal may have been previously cannulated.

### CROSS-REFERENCE TO RELATED APPLICATIONS

This application claims priority to U.S. Provisional Application No. 62/329,712 filed Apr. 29, 2016 and entitled “Animal Collar and Associated Systems and Methods,” which is hereby incorporated by reference in its entirety under 35 U.S.C. §119(e).

### GOVERNMENT SUPPORT

This invention was made with government support under Grant Nos. W81XWH-11-1-0561, W81XWH-16-1-0211 and W81XWH-16-1-0071, each awarded by the Department of Defense (USAMRAA). The government has certain rights in the invention.

### TECHNICAL FIELD

The disclosed technology relates generally to an animal restraint collar and in particular, to the devices, methods, and design principles allowing for the use of restrained use of unanesthetized animals in certain scientific applications.

### BACKGROUND

Multiple fields of research use mouse models for pupillometry, facial telemetry, and pain quantification via facial grimace. Current mouse restraint devices that do not require anesthesia fall short in allowing for facial analysis. Additionally, current mouse restraint devices do not allow for cannula injection in unanesthetized mice.

Thus, there is a need in the art for a better mousetrap: improved devices, systems and methods for restraining unanesthetized animals, including animals already having been cannulated. In exemplary implementations, the various devices, systems and methods here provide a two piece apparatus designed to restrain a mouse in the most minimal manner, without the use of anesthesia, and allow visualization of key facial features and other behavioral analyses.

### BRIEF SUMMARY

Discussed herein are various devices, systems and methods relating to an improved collar for use with certain laboratory animals. It is understood that prior art restraint devices that do not require anesthesia fall short in allowing for certain procedures, such as facial analysis.

One Example includes a collar for restraint of an alert, cannulated laboratory animal, including: a planar base, and a rectangular housing including a first end, a second end and at least one opening, where the housing defines a restraint chamber.

Implementations of this Example may include one or more of the following features. The collar where the base is substantially planar and includes paired base openings. The collar where the housing further includes mounting brackets defining bracket openings. The collar where the bracket openings are elongate bracket openings. The collar where the housing is configured to be secured to the base via fasteners disposed through the bracket openings into the base openings. The collar where the base and housing are substantially transparent. The collar further including a cannula. The collar where rectangular housing is configured to be in slidable communication with the planar base via the elongate bracket openings. The collar further including a cannula. The collar further including at least one recording device. The collar further including a cannula and at least one recording device. The collar where the planar base and rectangular housing are substantially transparent. The collar where rectangular housing further including mounting brackets defining elongate bracket openings. The collar where the housing is configured to be in slidable communication with the planar base via the elongate bracket openings. The collar further including a cannula. The collar where the base and housing are acrylic.

Another Example includes a collar system for use with a cannulated laboratory animal, including a planar base including paired base openings and a rectangular housing. The collar system also includes a lumen defining a restraint chamber. The collar system also includes at least one end opening. The collar system also includes paired mounting brackets including elongate bracket openings. The collar system also includes where the rectangular housing is configured to be secured to the base to restrain the animal via fasteners disposed through the elongate bracket openings and paired base openings.

Implementations of this Example may include one or more of the following features. The collar where rectangular housing is configured to be in slidable communication with the planar base via the elongate bracket openings. The collar further including a cannula. The collar further including at least one recording device. The collar further including a cannula and at least one recording device. The collar where the planar base and rectangular housing are substantially transparent. The collar where rectangular housing further including mounting brackets defining elongate bracket openings. The collar where the housing is configured to be in slidable communication with the planar base via the elongate bracket openings. The collar further including a cannula. The collar where the base and housing are acrylic.

Another Example includes a collar system for use with a cannulated laboratory animal, including a planar base including paired base openings, a rectangular housing including at least one end opening, the housing defining a restraint chamber, a cannula, and at least one recording device, where the rectangular housing is configured to be secured to the base to restrain the cannulated animal such that the body of the animal is disposed within the housing such that the head of the animal and cannula are disposed outside the housing via the at least one end opening. Implementations of this Example may include one or more of the following features. The collar where rectangular housing further including mounting brackets defining elongate bracket openings. The collar where the housing is configured to be in slidable communication with the planar base via the elongate bracket openings. The collar further including a cannula. The collar where the base and housing are acrylic.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is an endlong perspective view of the collar, according to one embodiment.

FIG. 2 is a three-quarters perspective view of the embodiment of FIG. 1.

FIG. 3A is an end schematic view of one implementation of the housing.

FIG. 3B is a top schematic view of the housing implementation of FIG. 3A.

FIG. 3C is a top schematic view of a base, according to one implementation.

FIG. 3D is a endlong perspective view of two implementations of the collar having a housing adopted for use with male mice on the left and a housing adopted for female mice on the right.

FIG. 4 is a top view of the collar having elongate bracket openings, according to one embodiment.

FIG. 5A is a perspective view of one implementation of the collar, wherein the housing has not yet been secured over the animal.

FIG. 5B is a perspective view of the collar of FIG. 5A, wherein the animal has been trapped within the housing.

FIG. 5C is a perspective view of the collar of FIG. 5A, wherein the housing is being oriented atop the base.

FIG. 6A is a perspective view of the collar according to one implementation, wherein the user is inserting a first fastener.

FIG. 6B is a is a perspective view of the collar of FIG. 6A, wherein the user is inserting a second fastener.

FIG. 6C is a perspective view of the collar of FIG. 6A, wherein the user is tightening the fasteners to secure the animal.

FIG. 6D is a perspective view of a secured animal inside the collar, according to the implementation of FIG. 6A.

FIG. 7A is a perspective side view of the collar system utilizing at least one recording device, according to one implementation.

FIG. 7B is a perspective end long rear view of the system of FIG. 7A.

FIG. 8A is a perspective close-up view of the collar system utilizing a cannulated animal, according to one implementation.

FIG. 8B is a perspective front view of the implementation of FIG. 8A.

FIG. 8C is a perspective side view of the collar system utilizing a cannulated animal, according to another implementation.

FIG. 9A is a perspective close-up view of the collar system, wherein the animal is undergoing eye-blink analysis, according to one implementation.

FIG. 9B is another perspective close-up view of the collar system, showing eye blink measurement, according to the implementation of FIG. 9B.

FIG. 9C is a perspective close-up view of the collar system, wherein the animal is undergoing eye-blink analysis during the control condition, according to one implementation.

FIG. 9D is another perspective close-up view of the collar system, showing eye blink measurement, according to the implementation of FIG. 9C.

### DETAILED DESCRIPTION

The various embodiments disclosed or contemplated herein relate to an animal restraint 10 or collar 10. In various implementations, the collar is 10 designed to minimally restrain a laboratory animal—such as a mouse, rat or other lab animal—in the most minimal manner without the use of anesthesia. This has implications for research using animal or mouse models in a variety of fields of scientific study and other applications.

As one non-limiting example, certain implementations allow for the visualization of key facial features and can be used in various studies, such as pupilometry, facial telemetry, and pain quantification by facial grimace. It is understood that myriad additional implementations are possible, and these would be appreciated by the skilled artisan.

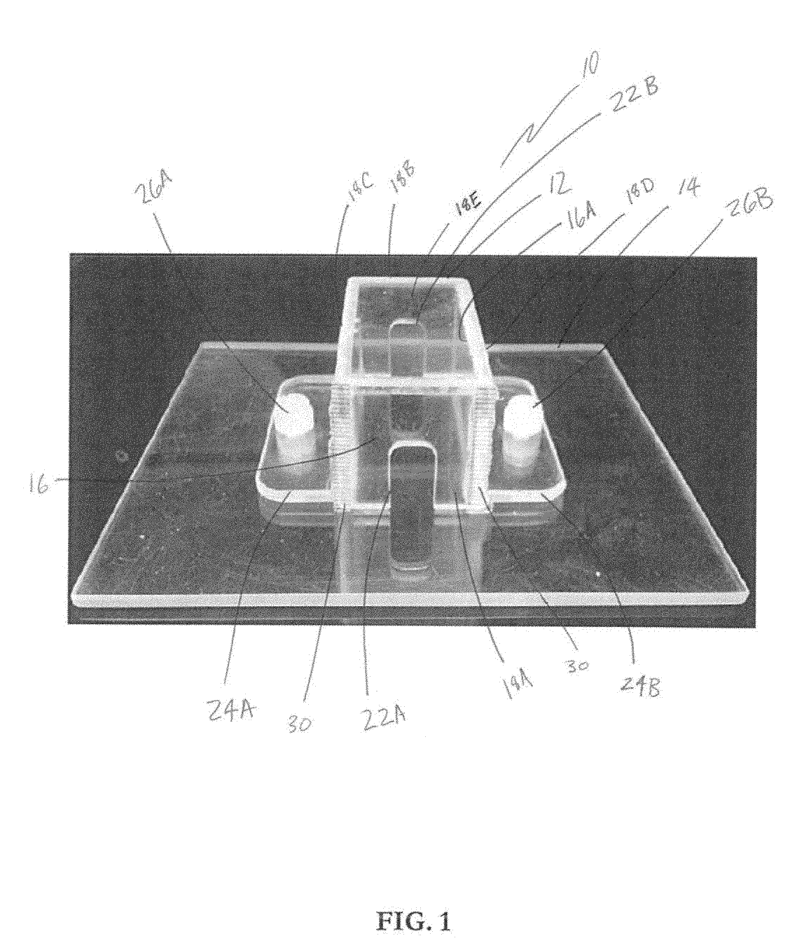

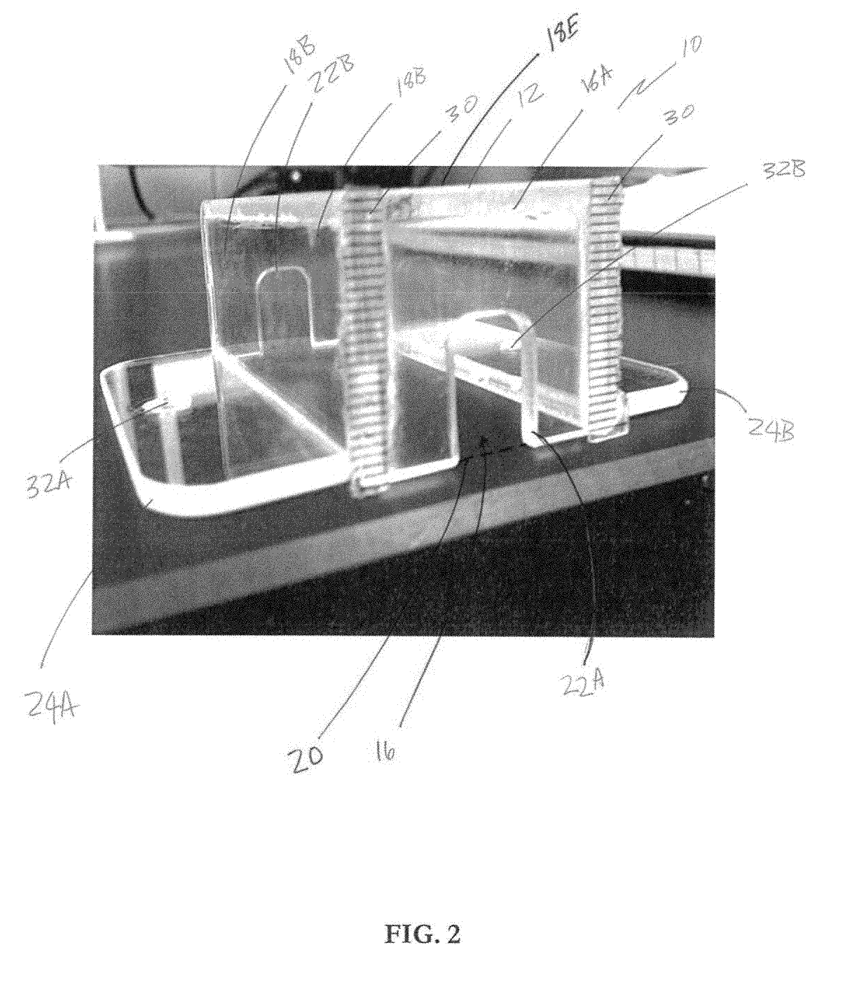

Turning to the drawings in greater detail, in the implementations of FIG. 1A-2 the collar 10 has a housing 12 that can be readily attached to a base 14. In the implementation of FIGS. 1-2, the housing 12 is a rectangular, elongate housing 12. The housing 12 defines an internal lumen 16 that is configured for animal confinement, and is also referred to herein as a restraint chamber 16.

The restraint chamber 16 in these implementations has an inner chamber surface 16A, and the housing has a plurality of chamber walls 18: a first end wall 18A and second end wall 18B, as well as a first side wall 18C and a second side wall 18D and an upper wall 18E, or roof 18E. In these implementations, the housing walls 18 are substantially planar and of sufficient strength and rigidity to confine the laboratory animal in the restraint chamber 16. It is understood that in this implementation the housing 12 therefore defines the walls on five of the six sides of the restraint chamber 16, and the planar base 14 defines the “floor” 20 of the restraint chamber 16. In use, and as described below, the housing 12 can therefore be used to “catch” and “trap” an alert animal placed on the base 14. It is understood that in alternate implementations, the housing 12 can have a floor (not shown).

Continuing with the implementations of FIGS. 1-2 and FIGS. 3A-3D, the housing 12 and base 14 can be fabricated from plastic, such as acrylic, or other rigid and durable materials known in the art. In certain implementations, the housing and/or base are transparent or translucent, though it is understood that transparency or translucency is not required for the collar 10 to function in all implementations.

In the implementations depicted in FIGS. 1A-3D, a first end opening 22A and second end opening 22B allow for the head and tail of the animal (not shown) to extend out of the first end 18 and/or second end 18B. It is understood that in various implementations, either opening 22A, 22B can accommodate the head or tail of the animal, as best shown in FIGS. 9A-D.

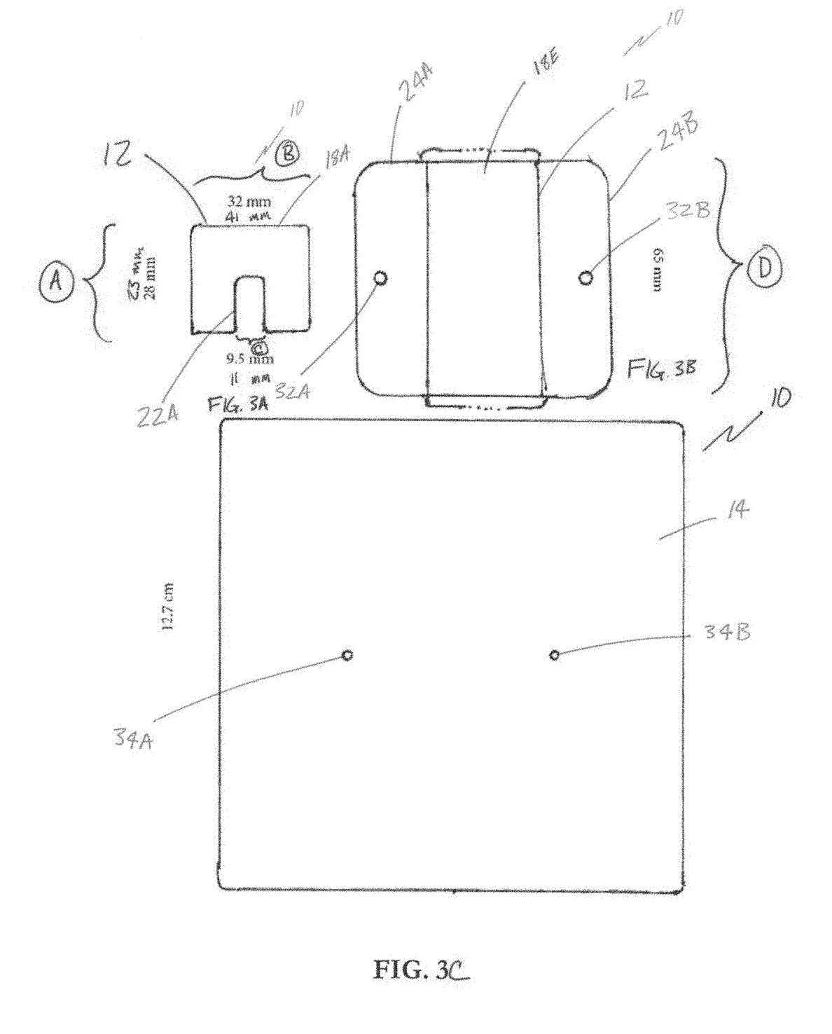

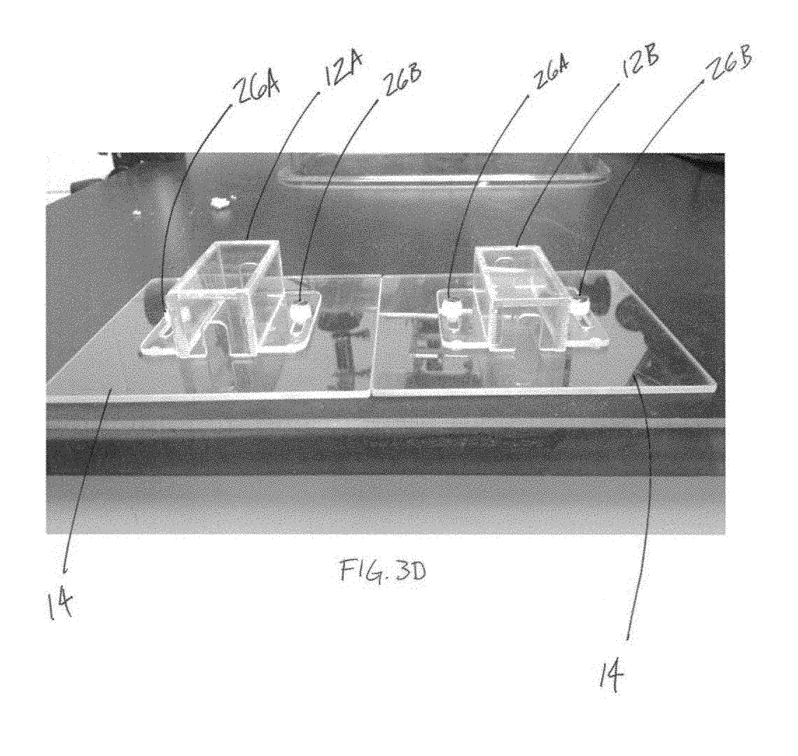

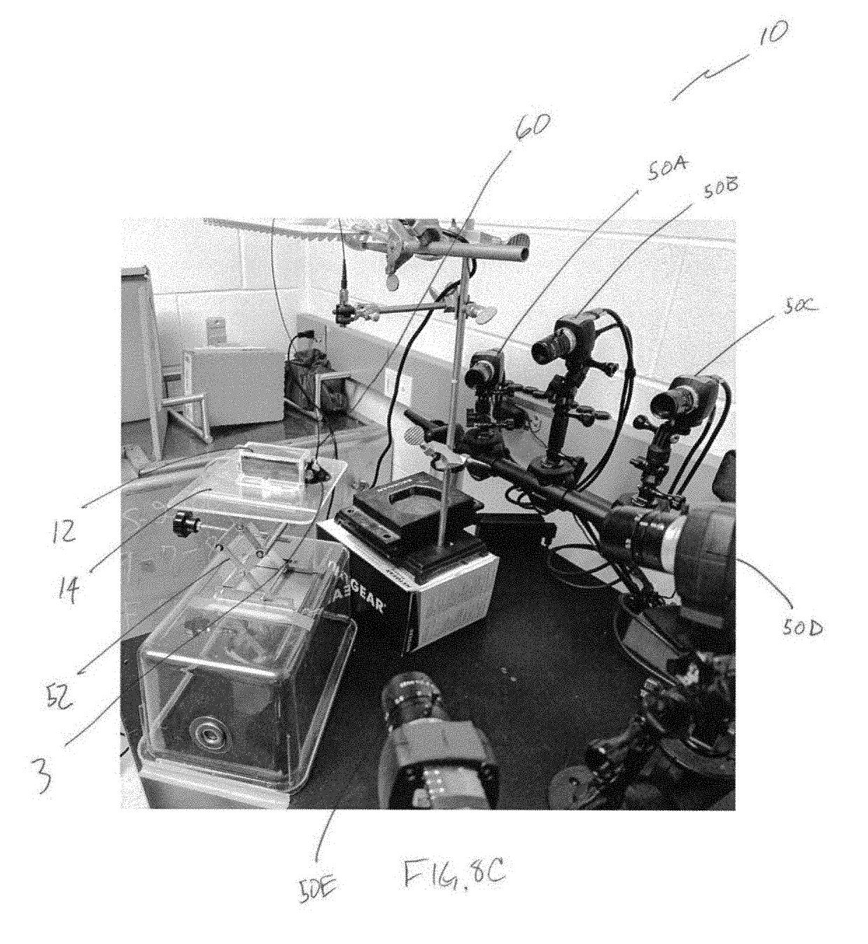

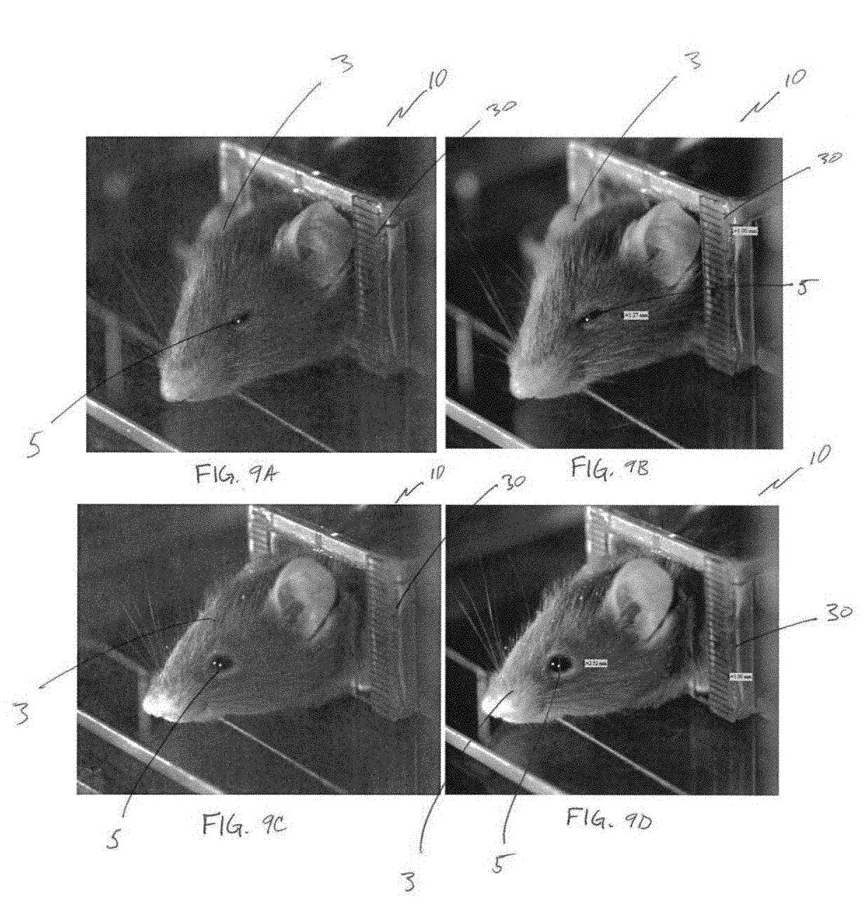

In the implementation of FIGS. 3A-3C, various housing 12 dimensions are possible. In certain implementations, and as shown in FIG. 2, hashings or markings 30 can be placed on the first end wall 18A and/or second end wall 18B to allow for ease in taking measurements of the animal. In certain implementations, numbers or other markings 30 can be used to define increments, such as millimeters. As is also shown in FIGS. 8A-8B, these markings 30 can be used to measure the animal 1 and its various movements, such as by way of a video camera, as shown for example in FIGS. 7A-7B at 50.

In an illustrative embodiment of the collar 10, and as shown in FIGS. 3A-3D, the dimensions of the chamber 16 were derived from the average dimensions of adult C57BL/6 mice, including skull diameter, neck width, shoulder width, body length, body width, and body height. It is understood that the chamber 16 can be scaled or sized variously for myriad animal types and dimensions.

Returning to the implementations of FIGS. 3A-D, in one non-limiting example of a housing 12 according to the implementations of FIGS. 3A-3D and configured for a female mouse can be about 23 mm tall (as shown in FIG. 3A at reference letter A), while a housing 12 for a male mouse can be about 28 mm tall. Similarly, a housing 12 width (reference letter B) of about 32 mm and opening 22A width (reference letter C) of about 9.5 mm can be used for non-obese mice, while a housing 12 width of about 41 mm and opening 22A width of about 11 mm can be used for obese mice. One of skill in the art would appreciate that the housing 12 may be about 65 mm long (reference arrow D), though again, these examples are intended to only be illustrative and can vary from a few millimeters to centimeters, decimeters or meters, depending on the species of animal 1 to be restrained. As shown in FIG.3D, in various implementations a male housing 12A and/or female housing 12B may be used in conjunction with various bases 14.

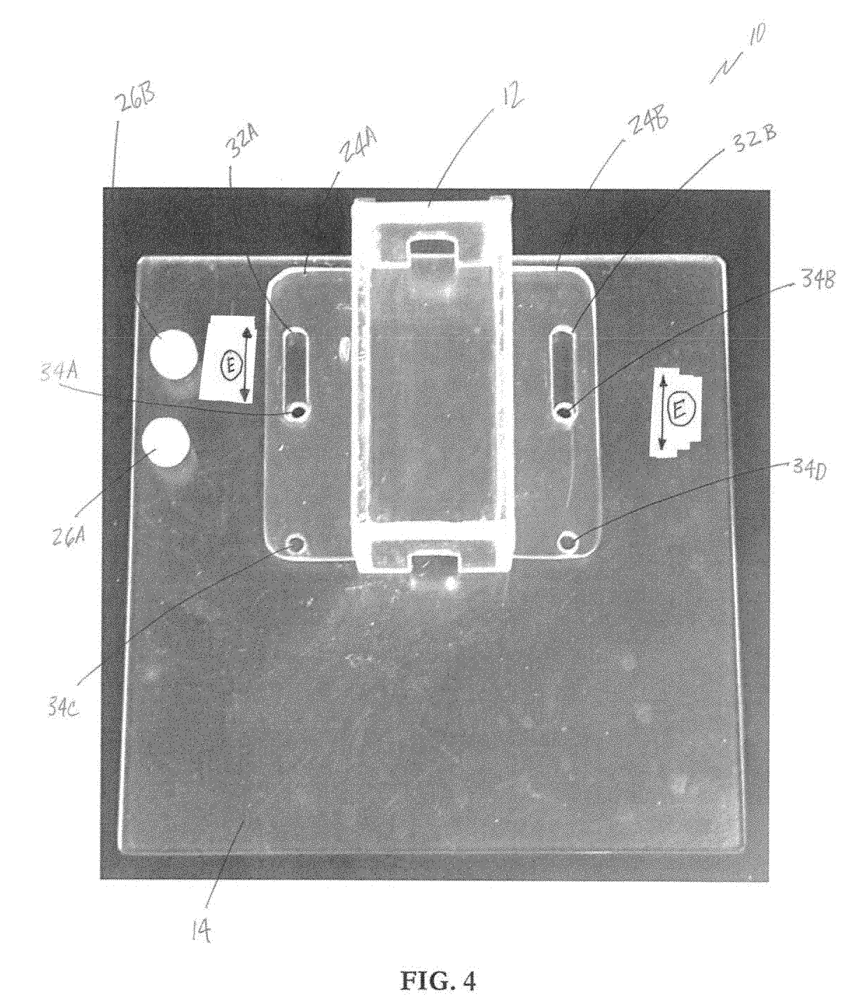

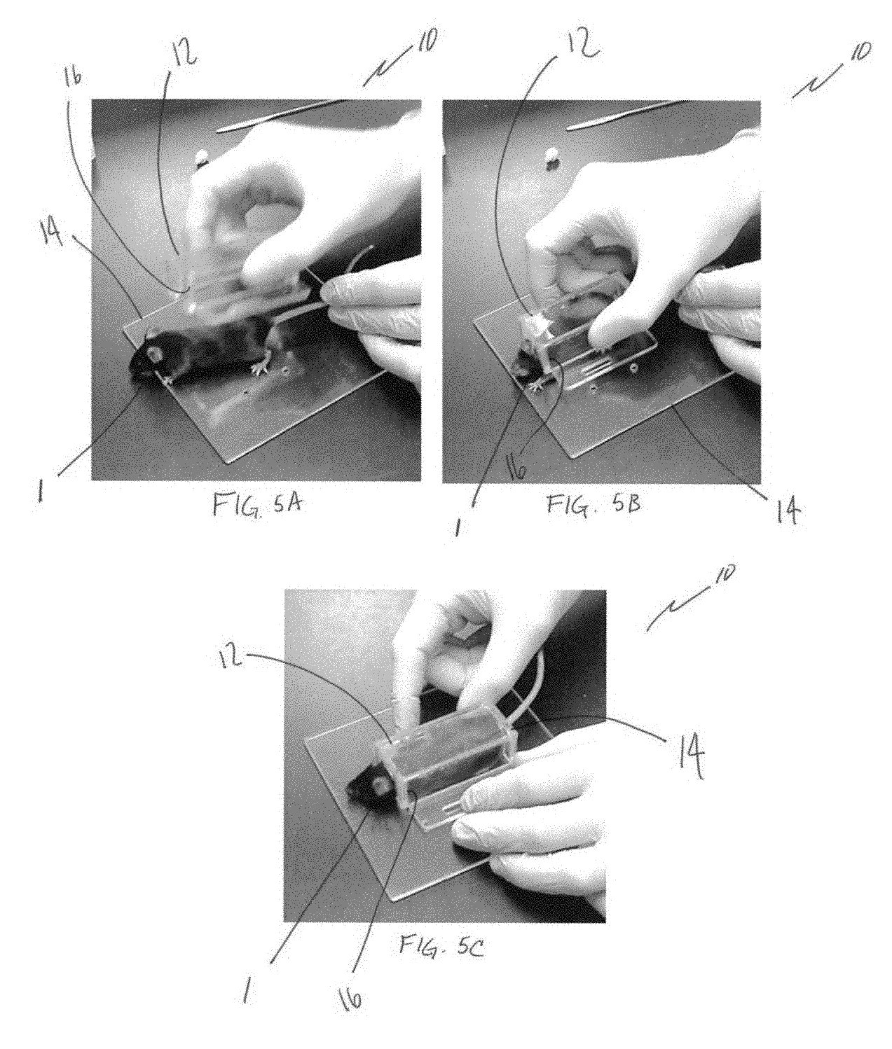

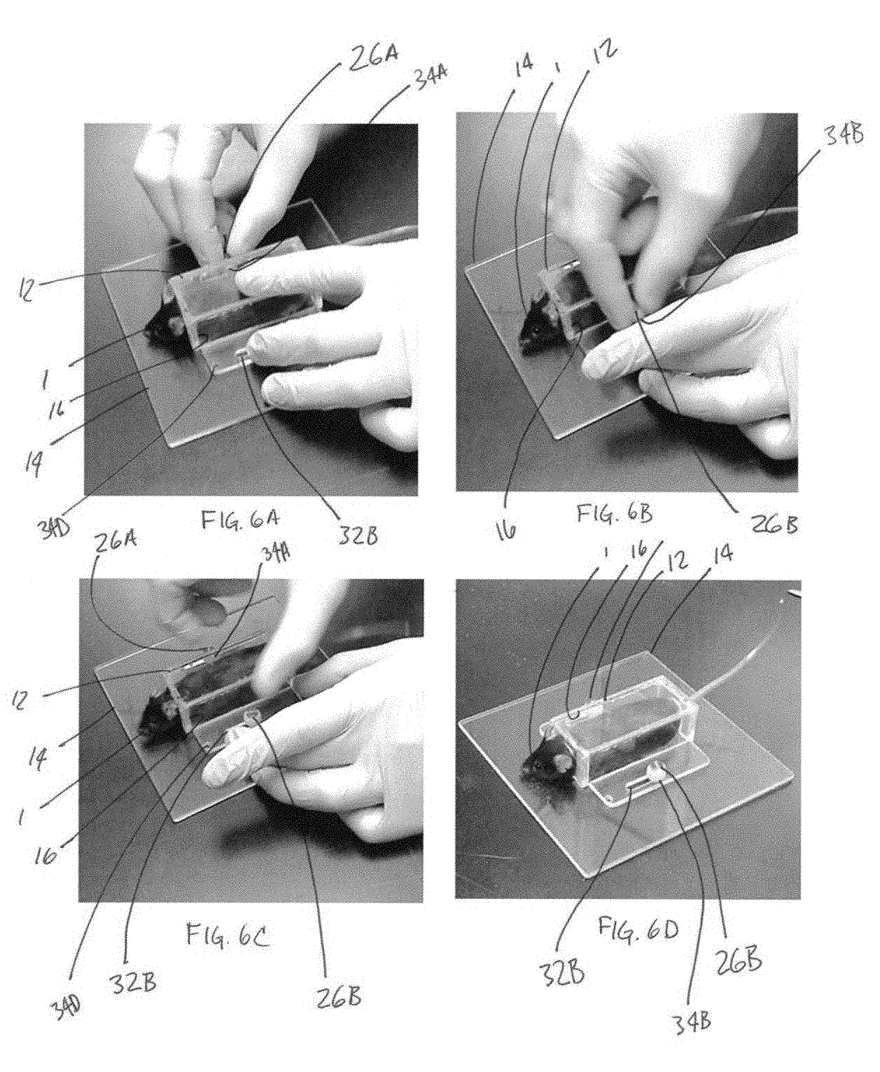

Continuing with the implementation of FIGS. 1A-3D, the collar 10 has mounting brackets 24A, 24B and fasteners 26A, 26B to selectively attach the housing 12 to the planar base 14, as best shown in relation to FIGS. 3A-4. As also best shown in FIG. 2-3C, the fasteners 26A, 26B—such as finger screws or other known bolts or fasteners—can be passed through bracket openings 32A, 32B and mounted in base openings 34A, 34B, as would be understood by a skilled artisan.

Turning to the implementation FIG. 4, the base 14 has a first paired set of base openings 34A, 34B and a second paired set of base openings 34C, 34D. Further, in this implementation, the mounting brackets 24A, 24B have elongate bracket openings 32A, 32B. It is understood that each of these features allow the user to re-position the housing 12 relative to the base 14, as shown at the reference arrows labeled E.

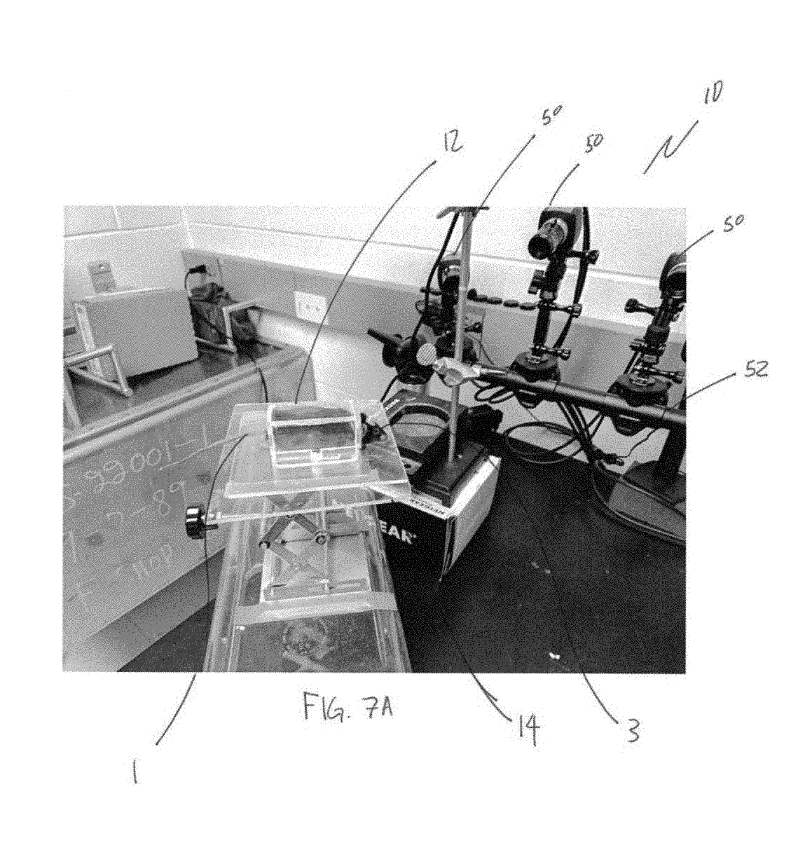

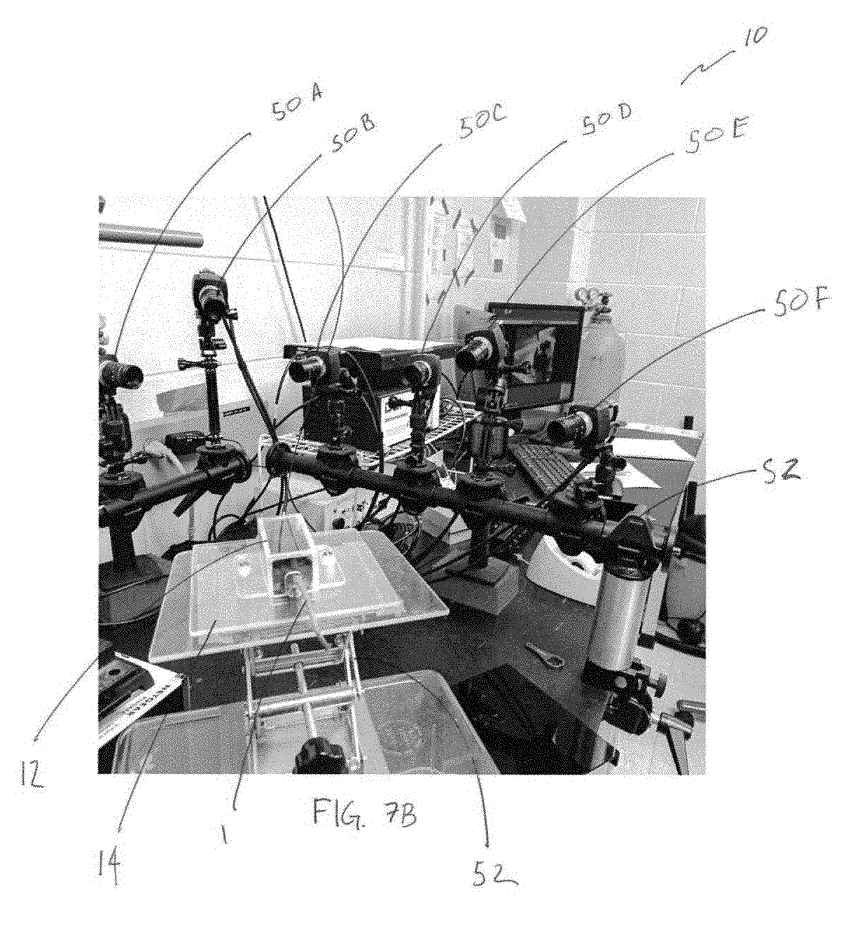

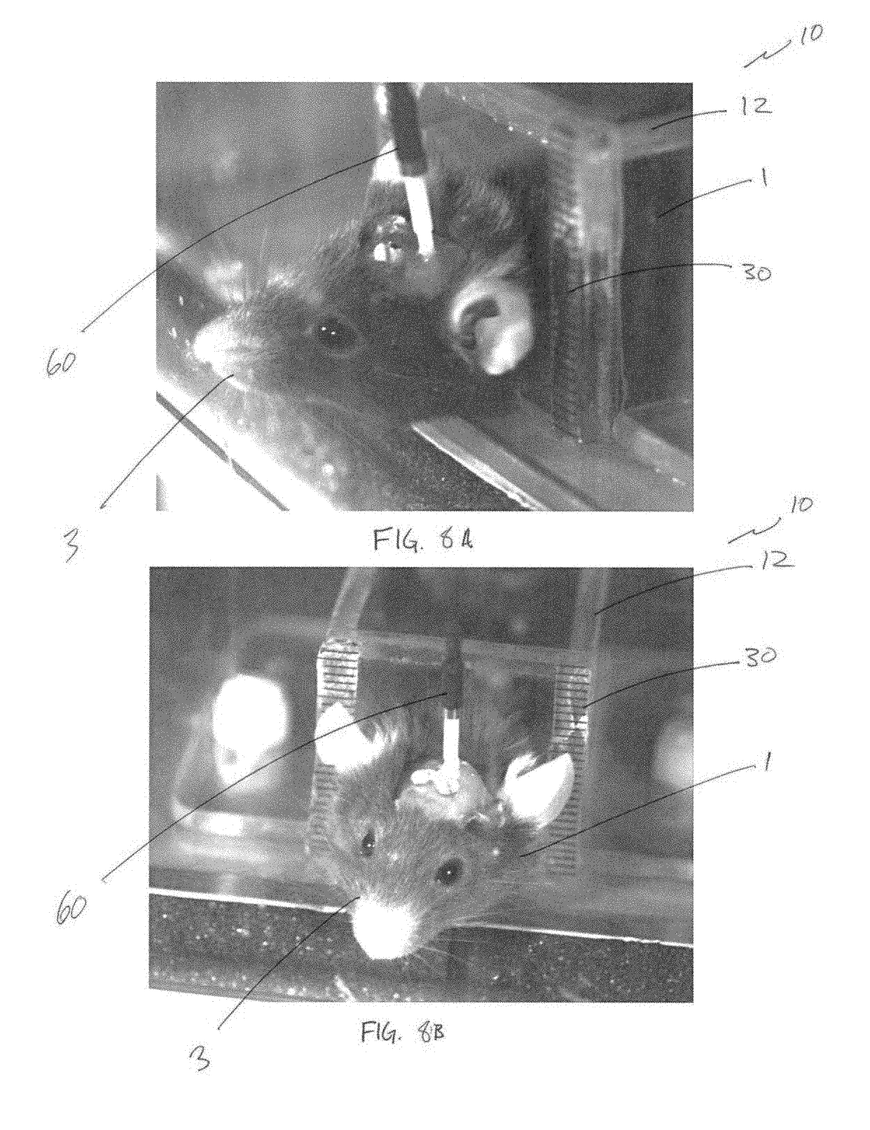

For example, in certain implementations the housing 12 can be re-positioned on a coarse level between various mounting positions with the various paired base openings 34A, 34B, 34C, 34D for use with distinct equipment, as shown generally at 100 in FIG. 6). It is further understood that the use of elongate bracket openings 32A, 32B permits the urging of the housing 12 along the base 14, for example by loosening the fasteners 26A, 26B.

In use, and as shown in the implementations of FIGS. 5A-6D, an animal 1 such as a mouse 1 can be placed on the base 14, and the user can bring the housing 12 down over the animal 1, as shown in FIG. 5A. It is understood that after the animal 1 has been secured inside the restraint chamber 16, the housing 12 can be positioned on the base 14, as is shown in the implementations of FIGS. 5B-5C.

As shown in the implementations of FIGS. 6A-6D, after securing the animal 1 inside the housing 12 restraint chamber 16 and orienting it on the base 14, the fasteners 26A, 26B can be inserted manually through the bracket openings 32A, 32B and secured into the applicable base openings 34A, 34B, 34C, 34D. Other methods of securing the housing 12 to the base 14 are possible, as would be appreciated by one of skill in the art.

In use, according to certain of the implementations and as also shown in FIGS. 5A-6D, one of skill in the art would appreciate that precautions can be taken to ensure the paws of the mice or other animals are not pinched while securing the housing 12. In on illustrative example, such precautions can include securing the housing 12 with only a quarter turn of the fasteners 26A, 26B. In certain implementations, this initial securing of the fasteners 26A, 26B can be performed while viewing paw placement from the underside of the base 14, followed by subsequent tightening only after the user has confirmed that all paws and digits are clear. Transparency of the material composing the housing 12 and/or base 14 is thus utilized in certain exemplary implementations. Further, transparent or translucent materials can also provide the user with the ability to monitor the animal's 1 respiratory rate.

In certain aspects, the animal 1 such as a mouse must first acclimate to being restrained in the chamber 16. In various circumstances, because the restraint of the chamber 16 is minimal, certain animals are able to rotate and push against the inner chamber surface 16A, ends 18A, 18B and walls 18C, 18D. In certain circumstances, after acclimation, the animal 1 typically resigns to the restraint and normally maintains the natural position.

In various implementations, the restrained animal 1 can have a range of motion with their head. In certain aspects, this range of motion can be full, except for “tucking.” In various implementations, the restraint chamber provides sufficient space such that the limbs of the animal are able to be freely moved inside the restraint 16.

As shown in the implementations of the collar 10 in FIGS. 7A-7B, the housing 12 and base 14 can be used to restrain or otherwise secure an animal 1 for analysis by way of at least one analytical or recording device 50, such as a camera 50. In various implementations, the animal 1 can be positioned such that the animal head 3 is exposed to the device 50. Further, as would be appreciated by one of skill in the art, in various implementations, certain additional structural components such as scaffolding 52 or the like can be disposed between the housing 12/base 14 unit and the recording devices 50. As shown in FIG. 7B, in certain implementations several recording devices 50A, 50B, 50C, 50D, 50E, 50F can be arrayed around the secured animal 1 so as to record from various angles.

As shown in FIGS. 8A-C, in certain implementations of the collar system 10, the animal 1 may be fitted with a cannula 60 or other implanted device 60 inserted directly into the head 3 of the animal 1. Critically, in these implementations, the collar 10 is capable of being fitted to the animal 1 after it has been cannulated 60, as shown above in FIGS. 5A-6D.

In various implementations of the collar 10, therefore, and as shown in FIGS. FIGS. 8A-C, the collar 10 allows a user to administer various drugs or other substances or stimuli into the brain of an otherwise fully awake or alert animal 1 via the cannula 60, such as a previously inserted cannula 60. It is understood that in certain implementations, use of the collar 10 on a previously habituated mouse 1—or other animal—can allow the user to avoid using anesthesia. It is further appreciated that elimination of anesthesia can present certain methodological advantageous, as anesthesia can confound results. Further, certain aspects can also allow the user to immediately monitor drug responses in the animal without waiting for any effects of anesthesia to subside.

In certain implementations, the at least one camera 50 can be used for the visualization of key facial features of the animal 1. In certain non-limiting implementations, the device 10 can allow for the user to record various kinds of data from the animal, such as orbital tightening, nose bulge, cheek bulge, and whisker position, for use in pain assessment, light sensitivity and other kinds of experiments known in the art or as yet developed. It would be understood by one of skill in the art that many additional experiments are possible and contemplated herein.

For example, in the collar 10 implementations of FIGS. 9A-9D, the animal 1 can be analyzed for eyeblink conditioning, including the measurement of eye 5 closure (shown in FIGS. 9A-9B) compared with normal (FIGS. 9C-D), by way of the recording devices 50 (shown in FIGS. 7A-C), with or without the application of a certain drug via the cannula. As would be understood by one of skill in the art, many experimental designs are contemplated.

Although the disclosure has been described with reference to preferred embodiments, persons skilled in the art will recognize that changes may be made in form and detail without departing from the spirit and scope of the disclosed apparatus, systems and methods.

What is claimed is:

1. A collar for restraint of an alert, cannulated laboratory animal, comprising:
a. a planar base; and
b. a rectangular housing comprising a first end, a second end and at least one opening, wherein the housing defines a restraint chamber.

2. The collar of claim 1, wherein the base is substantially planar and comprises paired base openings.

3. The collar of claim 2, wherein the housing further comprises mounting brackets defining bracket openings.

4. The collar of claim 3, wherein the bracket openings are elongate bracket openings.

5. The collar of claim 3, wherein the housing is configured to be secured to the base via fasteners disposed through the bracket openings into the base openings.

6. The collar of claim 1, wherein the base and housing are substantially transparent.

7. The collar of claim 1, further comprising a cannula.

8. A collar system for use with a cannulated laboratory animal, comprising:
a. a planar base comprising paired base openings; and
b. a rectangular housing comprising:
i. a lumen defining a restraint chamber;
ii. at least one end opening; and
iii. paired mounting brackets comprising elongate bracket openings,
wherein the rectangular housing is configured to be secured to the base to restrain the animal via fasteners disposed through the elongate bracket openings and paired base openings.

9. The collar of claim 8, wherein rectangular housing is configured to be in slidable communication with the planar base via the elongate bracket openings.

10. The collar of claim 8, further comprising a cannula.

11. The collar of claim 8, further comprising at least one recording device.

12. The collar of claim 8, further comprising a cannula and at least one recording device.

13. The collar of claim 8, wherein the planar base and rectangular housing are substantially transparent.

14. A collar system for use with a cannulated laboratory animal, comprising:
a. a planar base comprising paired base openings;
b. a rectangular housing comprising at least one end opening, the housing defining a restraint chamber;
c. a cannula; and
b. at least one recording device,
wherein the rectangular housing is configured to be secured to the base to restrain the cannulated animal such that the body of the animal is disposed within the housing such that the head of the animal and cannula are disposed outside the housing via the at least one end opening.

15. The collar of claim 14, wherein rectangular housing further comprising mounting brackets defining elongate bracket openings.

16. The collar of claim 15, wherein the housing is configured to be in slidable communication with the planar base via the elongate bracket openings.

17. The collar of claim 14, further comprising a cannula.

18. The collar of claim 14, wherein the base and housing are acrylic.

19. The collar of claim 14, wherein the base and housing are translucent.

20. The collar of claim 14, wherein the at least one recording device is at least one camera.
